(19) United States
(12) Patent Application Publication
(10) Pub. No.: US 2026/0259192 A1
(43) Pub. Date: Sep. 3, 2026

(54) Nanopore and Membrane Protein Multichannel Measurement Apparatus and Fabrication Method

(71) Applicant: Northeastern University, Boston, MA (US)

(72) Inventors: Meni Wanunu, Chestnut Hill, MA (US); Ali Fallahi, Chestnut Hill, MA (US); Amr Khalid Makhamreh, Chestnut Hill, MA (US)

(21) Appl. No.: 19/491,245

(22) PCT Filed: Jun. 12, 2024
(86) PCT No.: PCT/US2024/033686

§ 371 Date: Dec. 8, 2025

Related U.S. Application Data
(60) Provisional application No. 63/507,624, filed on Jun. 12, 2023.

(51) Int. Cl. G01N 33/487; C12Q 1/6869

(52) U.S. Cl. CPC G01N 33/48721; C12Q 1/6869

(57) ABSTRACT

Embodiments disclose a pore sensing fluidic device. The pore sensing fluidic device includes a structure defining a common inflow channel, shared channel with a plurality of apertures, a plurality of recording chambers, a common return channel, and a plurality of control channels arranged to support a fluid flow within the structure. The plurality of control channels connects the common inflow channel to the shared channel and the plurality of recording chambers. The fluidic device includes a sealer configured to isolate the plurality of recording chambers from the plurality of control channels. A membrane is formed and coupled to each of the plurality of apertures, and a pore is inserted into each membrane. A sensor, coupled to each of the plurality of apertures, is configured to produce a representation of flow through each of the plurality of membrane pores.

### RELATED APPLICATION

This application claims the benefit of U.S. Provisional Application No. 63/507,624, filed on Jun. 12, 2023. The entire teachings of the above application is incorporated herein by reference.

### BACKGROUND

In multiplexed nanopore systems, both the electrodes and electrolyte solution in either the primary (herein referred to as the common channel) or secondary (herein referred to as the return channel) fluid chamber must be completely isolated from the rest in order to preserve independent current recordings of every nanopore-containing membrane.

Currently, most nanopore array designs such as those used by legacy systems have electrically independent recording chambers, with an electrode embedded at the bottom of each chamber. Each chamber is subsequently insulated from the rest of the system once an organic membrane is formed at the mouth of the well. The dead-end well design has proven useful for tasks such as DNA and RNA sequencing. However, recent single-channel experiments (devices with only one membrane and one pore) have highlighted the utility of user access to both sides of a nanopore membrane device. For instance, addition of motor enzymes to one side of the system, while a protein sample is present in the opposite side of the system may allow for “pull-through” unfolding and translocation of the protein through the pore for measurements. Further, electrolyte and buffer imbalance across the two sides of the membrane allows for investigation of membrane protein or protein pore function and behavior, with application in both fundamental science and applied science, pharmaceutical development, and biotechnology.

### SUMMARY

Embodiments disclosed herein relate to a pore sensing fluidic device. The pore sensing fluidic device includes a structure defining a common inflow channel, a shared channel with a plurality of apertures, a plurality of recording chambers, a common return channel, and a plurality of control channels arranged to support a fluid flow within the structure, the plurality of control channels connecting the common inflow channel to the shared channel and the plurality of recording chambers. The fluidic device also includes a sealer configured to isolate the plurality of recording chambers from the plurality of control channels. The fluidic device also includes a membrane formed and coupled to each of the plurality of apertures, and a pore inserted into each membrane, a sensor, coupled to each of the plurality of apertures, configured to produce a representation of flow through each of the plurality of membrane pores.

In an embodiment, the sealer includes the plurality of control channels configured to have an electrical resistance higher than an electrical resistance of each of the plurality of recording chambers.

A further embodiment includes an electrode placed within each of the plurality of control channels, at least a subset of the electrodes configured to electrically insulate a recording chamber of the plurality of recording chambers.

In a further still embodiment, the at least a subset of electrodes placed within each of the plurality of control channels is further configured to have a voltage being equal to a voltage in the insulated recording chamber.

In another embodiment, the sealer includes a pressurized substance in combination with a sealing membrane, the pressurized substance forcing the sealing membrane to enter at least a subset of the plurality of control channels and isolate fluid flow from the shared channel and at least a subset of the plurality of recording chambers the plurality of recording chambers to the common inflow channel and the common return channel.

In another embodiment, the sealer includes a pressurized substance, the pressurized substance configured to enter at least a subset of the plurality of control channels and isolate fluid flow from the shared channel and at least a subset of the plurality of recording chambers to the common inflow channel and the common return channel.

In a further embodiment, the pressurized substance is non-soluble, non-conductive, and incapable of mixing with the fluid flow.

In an embodiment, the representation of flow through the membrane pores is a representation of a rate of the flow, a representation of a measurement of a substance in the flow, or a combination thereof.

In a further embodiment, the measurement of the substance in the flow includes representations of ionic flow, representations of molecules in the flow, or a combination thereof.

In a still further embodiment, the representation is processed by an external device configured to analyze the representation of a rate of flow, analyze a representation of the measurement of the substance in the flow, or a combination thereof.

Another embodiment includes the plurality of membranes formed on and coupled to each of the plurality of apertures having (i) an electrical resistance being infinite before the membrane comprises a pore, or (ii) the electrical resistance being determined by an ability of ions to flow through the pore in the membrane of the plurality of membranes.

In another embodiment, the pore is inserted into a membrane by introducing a fluid solution containing a protein into: (i) the shared channel, or (ii) each recording chamber of the plurality of recording chambers.

An embodiment further includes a shared channel input fluidic port configured to support fluid flow into the shared channel, and an output fluidic port configured to support fluid flow from the shared channel, a common inflow channel input fluidic port configured to support fluid flow into the common inflow channel, and an output fluidic port configured to support fluid flow from the common inflow channel, and a common return channel input fluidic port configured to support fluid flow into the plurality of recording chambers, and an output fluidic port configured to support fluid flow from the common return channel.

Another embodiment is directed toward a method of constructing a pore sensing fluidic device. The method includes defining, via a structure, a common inflow channel, a shared channel with a plurality of apertures, a plurality of recording chambers, a common return channel, and a plurality of control channels arranged to support a fluid flow within the structure, the plurality of control channels connecting the common inflow channel to the shared channel and the plurality of recording chambers. The method also includes associating a sealer with a plurality of control channels in an arrangement to selectively isolate the plurality of recording chambers from the plurality of control channels. The method further includes forming and coupling a membrane to each of the plurality of apertures and forming a pore in each membrane. The method includes coupling a sensor to each of the plurality of apertures and configuring the sensor to produce a representation of flow through each of the plurality of membrane pores. The method of constructing a pore sensing fluidic device disclosed herein may be configured to perform any of the functions or embodiments of the system, method, or apparatus claims disclosed herein.

Another embodiment is directed toward a pore sensing fluidic device for ascertaining a molecular makeup of a substance. The fluidic device includes means for isolating a portion of a fluid in a fluid flow, the portion being isolated in a chamber, and means for producing a representation of ionic current of or molecules in the portion of the isolated fluid through a pore at the chamber.

Another embodiment is directed toward a method of operating a pore sensing fluidic device for ascertaining a molecular makeup of a substance. The method includes isolating a portion of a fluid in a fluid flow, the portion being isolated in a chamber, and producing a representation of ionic current of or molecules in the portion of the isolated fluid through a pore at the chamber.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is an image of the general shape and structure of a linear segment of a multi-channel fluidic device.

FIG. 2 is a flow diagram for a method of constructing a fluidic device, according to an embodiment.

FIG. 3 is two schematic diagrams of an equivalent electrical circuit of the fluidic device, according to an embodiment.

FIGS. 4A and 4B are lateral cross sections of a valve-sealed device, according to an embodiment.

FIG. 5 is a top view of a fluidic device in the open configuration, according to an embodiment.

FIG. 6 is a top view of a valve-sealed device in the sealed configuration, according to an embodiment.

FIGS. 7A and 7B are lateral cross sections of a fluid-sealed device, according to an embodiment.

FIG. 8 is a top view of a fluidic device in the open configuration, according to an embodiment.

FIG. 9 is a top view of a valve-sealed device in the sealed configuration, according to an embodiment.

FIG. 10 is an image depicting formation of membrane via droplet flow in the shared channel, according to an embodiment.

FIGS. 11A and 11B are lateral cross sections of a resistively insulated device, and the equivalent electrical circuit, according to an embodiment.

FIG. 12 is a top view of a resistively insulated device.

FIGS. 13A and 13B are lateral cross sections of a resistively insulated device comprising shield electrodes, and the equivalent electrical circuit, according to an embodiment.

FIG. 14 is a top view of a resistively insulated device with shield electrodes.

### DETAILED DESCRIPTION

A description of example embodiments follows.

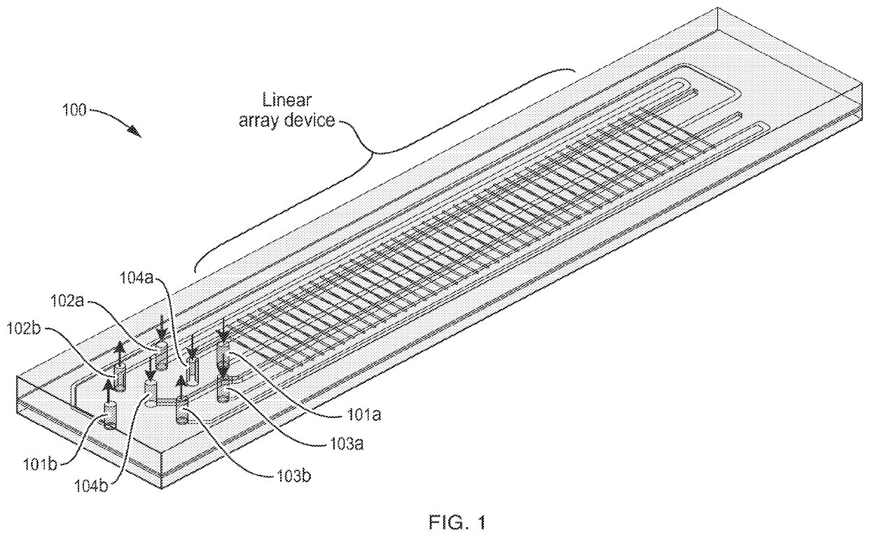

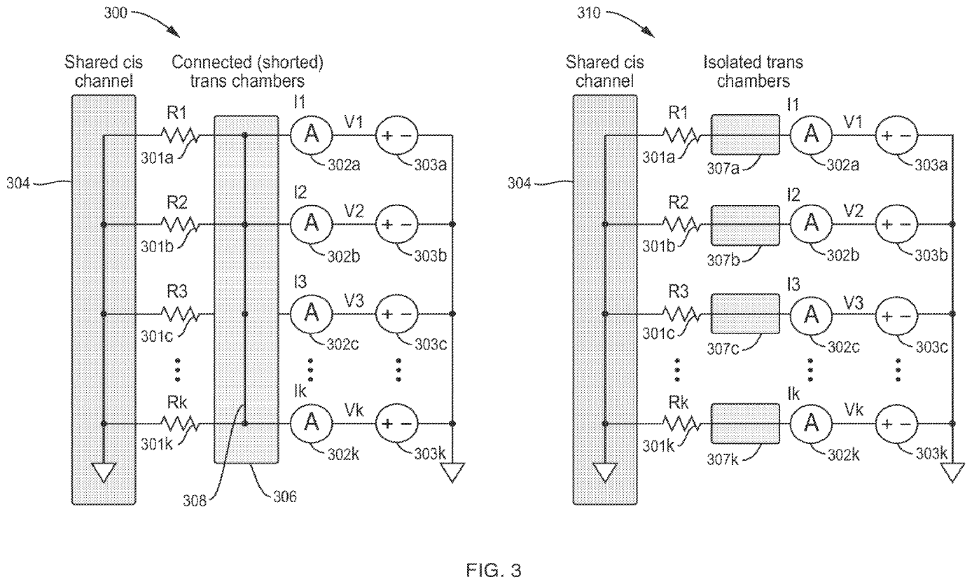

Embodiments disclose a platform that relates to the field of nanopore sensing, membrane protein analysis, membrane materials characterization, and membrane interactions with other organic or inorganic materials. Embodiments implemented herein relate to a versatile platform, i.e., a fluidic device, that provides the user the option to test different combinations of membranes, pores, and electrolyte solutions on a nanopore array that can also be replenished with fresh nanopore sensors once the previous set has expired. The design principle disclosed herein is dynamic separation/joining of fluid channels during an experiment (electrical equivalent circuit described below, at least in reference to FIG. 3 below). Embodiments disclose methods to reversibly insulate all recording chambers from the common inflow channel and the common return channel on command. The general outline of a device equipped with these methods is shown below, at least in reference to FIG. 1.

Simultaneous user access to both the shared channel and the recording chamber on opposite sides of a membrane has not been implemented on a multiplexed device. The main challenge of multiplexing and scaling up a system with both shared channel and the recording chamber is how to separate channels into independent recording units, while allowing for flow of solution (user access). Embodiments disclosed herein provide for these benefits.

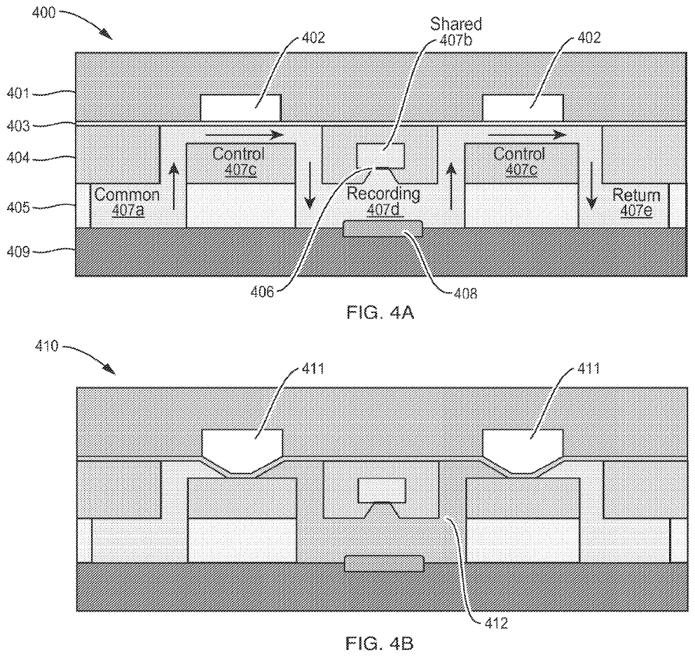

FIG. 1 is an image of the general shape and structure of a linear segment of a multi-channel fluidic device 100, according to an embodiment. One of the advantages of the embodied fluidic device is its ability to separate channels into independent recording units, while allowing for flow of solution. The fluidic device allows the user to isolate recording chambers from the common inflow and common return channels, as well as apply a membrane containing material and a solution containing the molecules to be analyzed in proximity to the isolated recording chambers. The fluidic device 100 includes the shared channel input port 101a, and shared channel output port 101b. The shared channel may be the channel which a user of the fluidic device may insert droplets containing membrane material as well as the solution containing molecules to be analyzed. In addition, the fluidic device 100 includes a common inflow channel input port 102a and common return channel input port 103a, as well as respective common inflow channel output port 102b and common return channel output port 103b. These sets of input and output ports may be used by an end user, or a fluid flow controller machine, to insert a fluid throughout the fluidic device. Once the fluidic device has had all fluids added, utilization of the input valve control port 104a and the output valve control port 104b allows a pressurized substance (such as a gas or a fluid) to be inserted into the system. When the pressurized substance is introduced, it isolates the recording chamber within the fluidic device by sealing it from the common inflow and common return channels. Once all fluids have been added and the respective recording chambers have been isolated, a membrane is formed within the shared channel and a pore is introduced into the membrane. The substance to be analyzed may flow downward to an electrode, which will analyze the substance. Further description of the channels and isolation embodiments is described below, at least in reference to FIGS. 4A-FIG. 10.

There are several drawbacks with legacy systems that may be solved by embodiments disclosed herein. For example, while legacy systems are currently implemented for DNA and RNA nanopore sequencing, there are design constraints that are insufficient in overcoming persistent challenges in nanopore sensing. First, the electrolyte solution in the recording chamber is inaccessible to the user, limiting the scope of experimental configurations possible. Second, the flowcell containing the nanopore array must be packaged and shipped wet to the user, i.e., the nanopore array device must be loaded with the electrolyte solution, membranes must be formed to insulate the shared channel from the recording chamber, and single nanopores must be inserted into the membrane of each channel. Hence, nanopore channels that make up the array are likely to become unstable after long-term storage. Moreover, the user cannot swap out and test different combinations of membranes and nanopores on the array. And third, the sensing lifetime of each nanopore in the array can end due to various factors such as irreversible clogging, ejection from the membrane, and membrane leakage or breakdown. Under optimal conditions, a legacy system sequencing experiment may last up to 72 hours, however, after the first 24 hours, approximately 50% of the pores remain active in the array.

Due to these time-dependent circumstances, maximizing the sensing throughput with some legacy system nanopore array requires samples to be loaded at high concentrations where only one in one million molecules are analyzed. Additionally, legacy shared channel design, which encapsulates the full surface area of the nanopore array, presents a challenge for single cell nanopore sequencing because it has an input volume of several hundred microliters. At this scale low-concentration analytes have a low probability of being captured and sensed, since their transit towards the proximity of the pores is diffusion-limited. Embodiments disclose a platform that overcomes these design constraints may benefit the field of nanopore sensing, membrane protein analysis, membrane materials characterization, and membrane interactions with other organic or inorganic materials.

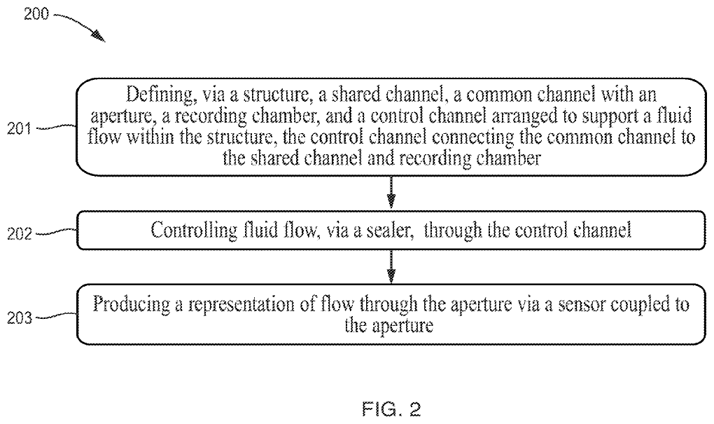

FIG. 2 is a flow diagram 200 for a method of constructing a fluidic device, according to an embodiment. The method includes defining 201 a common inflow channel, a shared channel with a plurality of apertures, a plurality of recording chambers, a common return channel, and a plurality of control channels arranged to support a fluid flow within the structure. The plurality of control channels may run perpendicular to and connect the common inflow channel to the shared channel and the recording chambers. As disclosed in relation to FIG. 1, the fluidic device may also include a shared channel input fluidic port configured to support fluid flow into the shared channel, and an output fluidic port configured to support fluid flow from the shared channel, as well as a common inflow channel input fluidic port configured to support fluid flow into the common inflow channel, and an output fluidic port configured to support fluid flow from the common inflow channel, a common return channel input fluidic port configured to support fluid flow into the common return channel, and an output fluidic port configured to support fluid flow from the common return channel. The plurality of control channels may run perpendicular to and connect the common inflow and common return channels to the plurality of recording chambers.

The method of construction the fluidic device also includes controlling 202 fluid flow through the plurality of control channels via a sealer. In some embodiments, the sealer utilizes a pressurized substance, such as a gas, in combination with a membrane. The pressurized gas forces the membrane to enter each of the plurality of control channels and isolate fluid flow from the shared channel and recording chambers to the common inflow channel and the common return channel (See FIGS. 4A and 4B). In other embodiments, the sealer utilizes a pressurized substance, such as a fluid. The pressurized fluid enters the plurality of control channels and isolates fluid flow from the shared channel and plurality of recording chambers to the common inflow channel and the common return channel. Isolating the recording chambers allows the analysis of the solution to be performed. When the pressurized substance is a fluid, the fluid may be non-soluble, non-conductive, and incapable of mixing with the fluid flow.

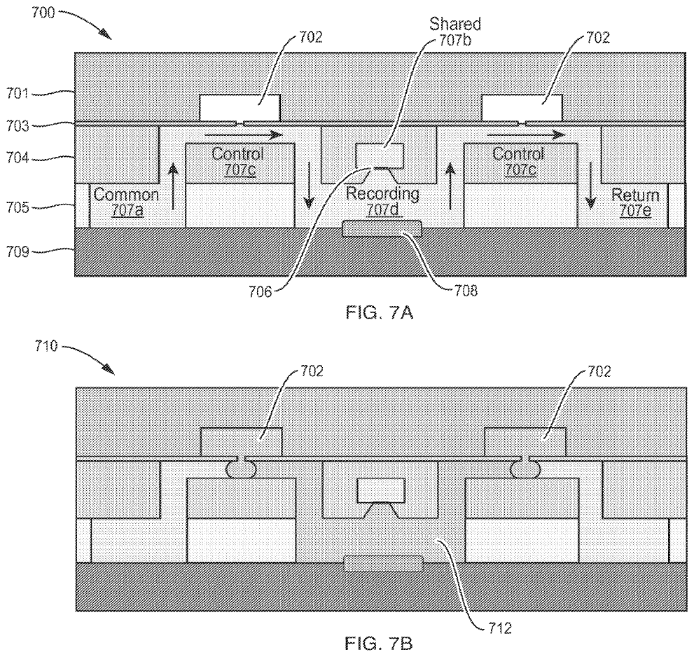

Still referring to FIG. 2, the method of construction the fluidic device also includes producing 203 a representation of flow through the plurality of apertures via an individual sensor coupled to each aperture for analysis. In these embodiments, an aperture of the plurality may be located within the shared channel, positioned above a recording chamber of the plurality(See FIGS. 4A and 4B, and 7A and 7B). The representation of flow through the aperture may be a representation of a rate of the flow, a representation of a measurement of a substance in the flow, or a combination thereof. In some embodiments, the measurement of the substance in the flow includes representations of ionic flow, or representations of molecules in the flow. The representation obtained may be processed by an external device configured to analyze the representation of a rate of flow or analyze a representation of the measurement of the substance in the flow. A membrane may be formed and coupled to each aperture of the plurality of apertures. The membrane may have (i) an electrical resistance being infinite (infinite electrical resistance may be defined as being at least ten times the electrical resistance of an inserted pore up to an electrical resistance approaching infinity). when the membrane does not comprise a pore, or (ii) the electrical resistance being determined by an ability of ions to flow through the pore in the membrane.

FIGS. 3A and 3B show schematic diagrams 300 and 310 respectively of an equivalent electrical circuit of the fluidic device. Referring to diagram 300 of FIG. 3A, each membrane aperture is shown as a resistor 301a-k. For channels corresponding with resistors 301a-n, where the resistors represent the electrical resistance of a membrane in the shared channel above each recording chamber, the current I₁ through Iₖ (302a-k) is measured under the applied voltages V₁ through Vₖ (303a-k). When no membrane is present, the resistance at the aperture is low. When a membrane is fully formed, the resistance is infinite, and when a pore is inserted into the membrane, the resistance of the pore may be determined by the ability of ions to flow through it, providing the sensing functionality. As shown in schematic diagram 300, the recording chambers 306 are connected to the shared channel 308 (during flow phase). These chambers 306 are effectively short-circuited and cannot be recorded independently at this stage.

Referring to the schematic diagram 310 of FIG. 3B, when the recording chambers 307a-k are insulated from the shared channel 308, i.e., a pressurized substance such as the gas and membrane combination or a pressurized fluid have entered the control channels and isolated the recording chamber, each recording chamber 307a-k may provide an independent measurement of current, corresponding to its own membrane and inserted pore.

In an embodiment, each nanopore chamber in the device is electrically insulated using dynamically actuated control valves. First, a common inflow channel delivers the electrolyte solution to the nanopore sensing chambers within the array via control channels (described below, at least in reference to FIGS. 4A and 4B and FIGS. 7A and 7B disclosed below). The walls for the control channels and recording chambers are fabricated onto a substrate using a photoresist (PR) material, such as a dry film PR, that may be patterned with techniques like photolithography (PL) or electron-beam lithography (EBL). At the bottom of each recording chamber may be an electrode for measuring the ionic current through the pore, where the electrode is a metallic pad routed to an amplifier. An aperture support for downstream membrane formation may be fabricated at the top of each recording chamber by patterning a laminated dry film PR layer that covers the control channels and sensing chambers via PL or EBL. Buried vias may be patterned into the dry film layer for delivery of electrolyte solution into the recording chamber from the common inflow and common return channels. Additional dry film layers may be laminated on top of the dry film layer that forms the aperture supports to fabricate a shared channel encapsulating the aperture supports or assist in routing fluids in any of the channels. Walls for the control channels may also be patterned into the additional dry film layers using PL or EBL. An elastomeric layer may be fastened on top of the last dry film layer to cover the control channel walls. Finally, a top substrate with cavities may be placed on top of the elastomeric layer and positioned to have the cavities on top of the control patterned on the last dry film layer.

FIGS. 4A and 4B are lateral cross sections of a valve-sealed fluidic device illustrating its structure and operating principle, according to an embodiment. FIG. 4A shows the open configuration 400 of the valve-sealed fluidic device. The fluidic device includes a top substrate 401. The control gas 402 is positioned above the elastomer 403 in this configuration, as the flow channel is open. The fluidic channels are contained within the dry film resist 404 and the channel walls 405 all of which is positioned above the bottom substrate 409. The fluidic device also includes the common inflow channel 407a, within the buffer as well as a shared channel 407b, a control channel 407c, a recording chamber 407d and a common return channel 407e. The control channels are connected to the recording chamber, and the fluid may flow through.

In FIG. 4B, the fluidic device 410 is the same as the fluidic device 400, however the control valve elastomer 403 is pressurized with a control gas 411, which creates a bulge to seal and disconnect each recording 407d chamber from the common inflow channel 407a, and the common return channel 407e by restricting fluid flow through the control channels 407c, creating an electrically insulated recording chamber 412.

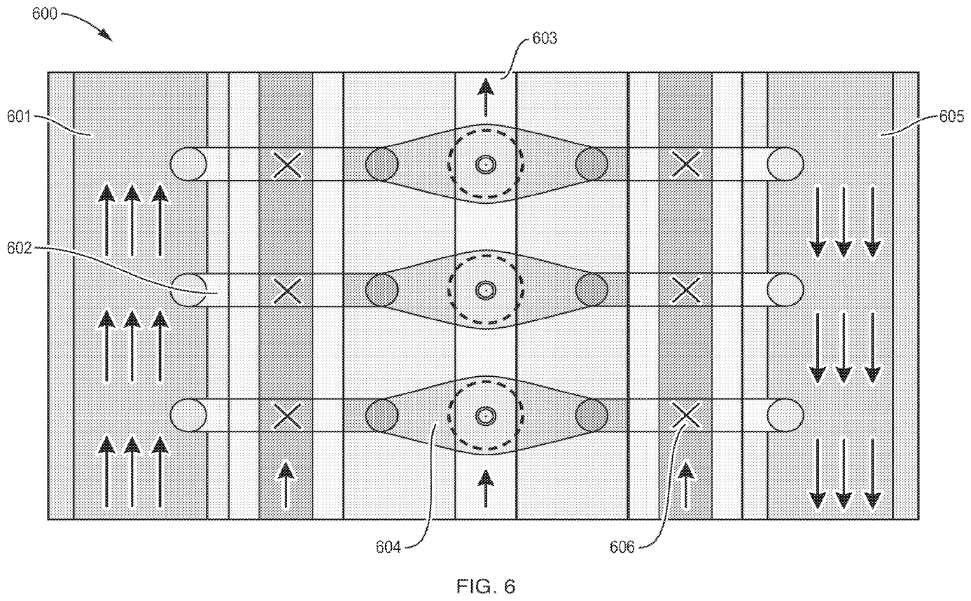

The current through a nanopore is independently recorded once the recording chambers electrically insulated from the common inflow and common return channels. Each recording chamber for nanopore sensing may consist of the following: (i) an aperture support for downstream membrane formation, (ii) an organic membrane or a biological membrane. At first, all recording chambers are electrically connected and incapable of being recorded individually. After flow of desired solution passes though all recording chambers, each control is then sealed to separate each nanopore sensing chamber (recording channel) from the common inflow and common return channels. The control channels are disconnected from the common inflow and common return channels when, according to an embodiment, an elastomeric layer adjacent to the control channels are compressed or decompressed upon the application of an external force, such as pressurized air introduced into cavities within the top substrate. As a result, the control channels are fully sealed from the common inflow and common return channels, allowing for each nanopore in the array to be electrically insulated (see FIG. 4B above, and FIG. 6 below). The implementation of an elastomeric layer allows electrical insulation of the recording chambers to be a reversible process. Therefore, the aperture supports can be reused for nanopore sensing experiments, enabling different electrolytes and samples to be run on the same nanopore array.

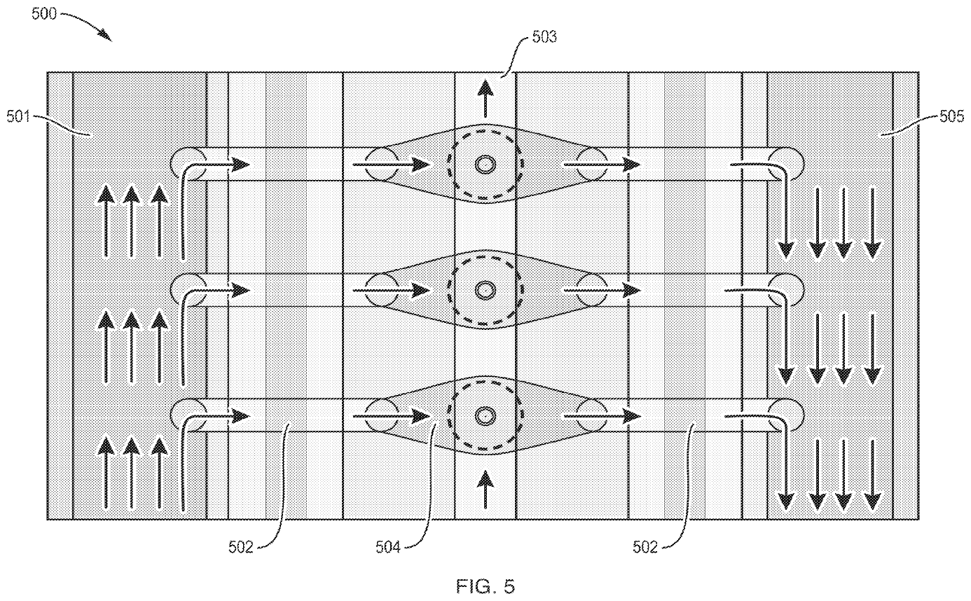

FIG. 5 is a top view 500 of the fluidic device in the open configuration, according to an embodiment. Since this fluidic device is in the open configuration, i.e., the pressurized gas is not pressing on the membrane, fluid can flow through all channels as in configuration 400 of FIG. 4A. Stated further, fluid can flow from the common inflow channel 501, the control channel 502, the shared channel 503, the recording chamber 507, and the common return channel 508.

FIG. 6 is a top view 600 of a valve-sealed device in the sealed configuration, according to an embodiment. The fluidic device 600 is the same as the fluidic device 500, it includes the common inflow channel 601, control channel 602, shared channel 603, recording chamber 604 and the common return channel 605, however the control valve elastomer over the control channels 602 has been pressurized with a control gas. This pressurization creates sealed 606 recording chambers 604 and a sealed shared channel 603.

In another embodiment, fabrication is similar to the previous embodiment with the exception of the elastomer layer. Instead of actuation of elastomeric materials sealing the control channels, the control channels can be separated from the common and common return channel with stable insulating droplets (made of gas or non-mixing, non-conductive liquids such as oils or fluorocarbons), according to an embodiment, that can be positioned within the control channels to fully seal them from the common and common return channel. (See FIGS. 7A and 7B, FIG. 8, and FIG. 9) These microdroplets can be kept fixed within the control channels when injected into a through-hole from the shared channel. Like the elastomer-based sealing design, the microdroplets can be removed and introduced into the control channels repeatedly, allowing for different electrolytes and samples to be run on the nanopore array.

FIGS. 7A and 7B are lateral cross sections of a fluid-sealed fluidic device illustrating its structure and operating principle, according to an embodiment. FIG. 7A shows the open configuration 700 of the fluid-sealed fluidic device. The fluidic device includes a top substrate 701. The sealing fluid channel 702 is positioned above the elastomer 703 in this configuration, as the flow channel is open. The pressure in the sealing fluid 702 is controlled to prevent droplet introduction into the control channels and to keep the control channels open. The fluidic channels are contained within the dry film resist 704 and the channel walls 705 all of which is positioned above the bottom substrate 709. The fluidic device also includes the common inflow channel 707a, within the buffer as well as a shared channel 707b, a control channel 707c, a recording chamber 707d and the common return channel 707e. The control channels are connected to the recording chamber, and the fluid may flow through.

In FIG. 7B, the fluidic device 710 is the same as the fluidic device 700, however the elastomer 703 has a pressurized sealing fluid 711 is injected as a microdroplet, which insulates and disconnects each recording 707d chamber from the common inflow channel 707a and the common return channel 707e, creating an electrically insulated recording chamber 712.

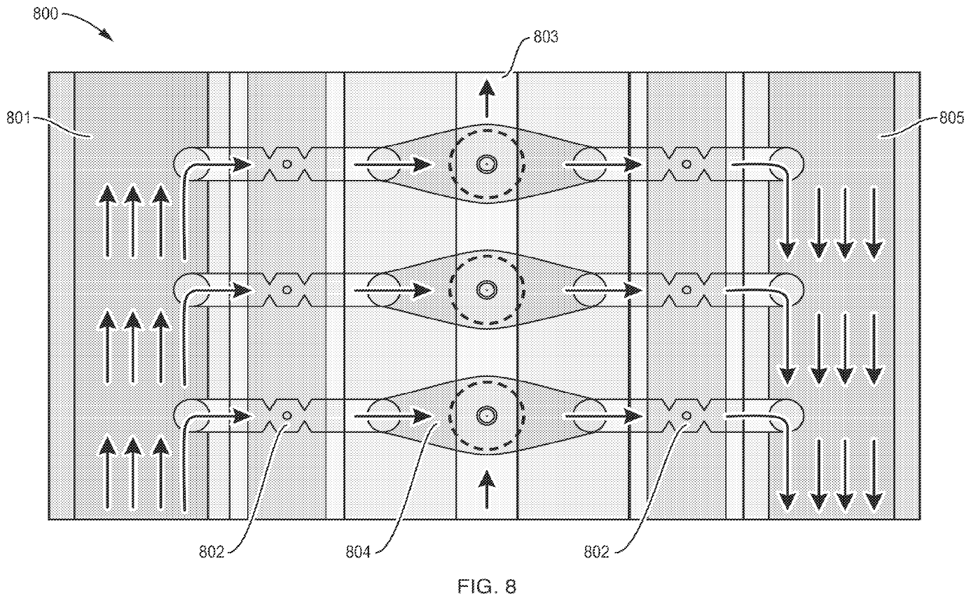

FIG. 8 is a top view 800 of the fluidic device in the open configuration, according to an embodiment. Since this fluidic device is in the open configuration, i.e., the pressurized fluid or gas has not been activated in the system, therefore fluid can flow through all channels as in configuration 400 of FIG. 4A or 700 of FIG. 7A. Stated further, fluid can flow from the common inflow channel 801, the control channel 802, the shared channel 803, and the recording chamber 807, and the common return channel 808.

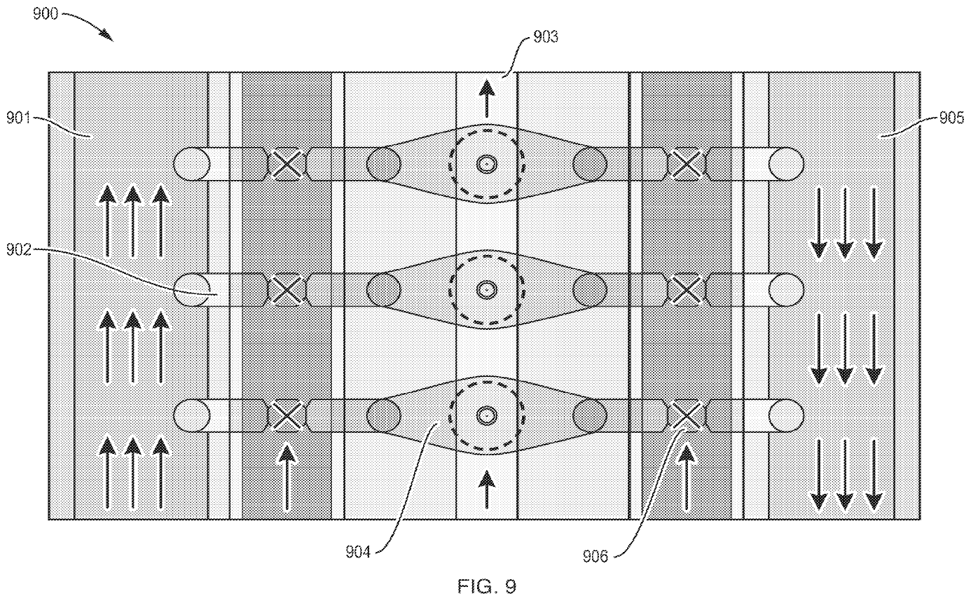

FIG. 9 is a top view 900 of a sealed fluidic device in the sealed configuration, according to an embodiment. The fluidic device 900 is the same as the fluidic device 800, it includes the common inflow channel 901, control channel 902, shared channel 903, recording chamber 904 and common return channel 905. However, the control valve elastomer over the control channels 902 has been injected with a sealing fluid, or a pressurized gas has created a bulge in the membrane, depending on the embodiment. This sealing creates sealed 906 recording chambers 904 and a sealed shared channel 903.

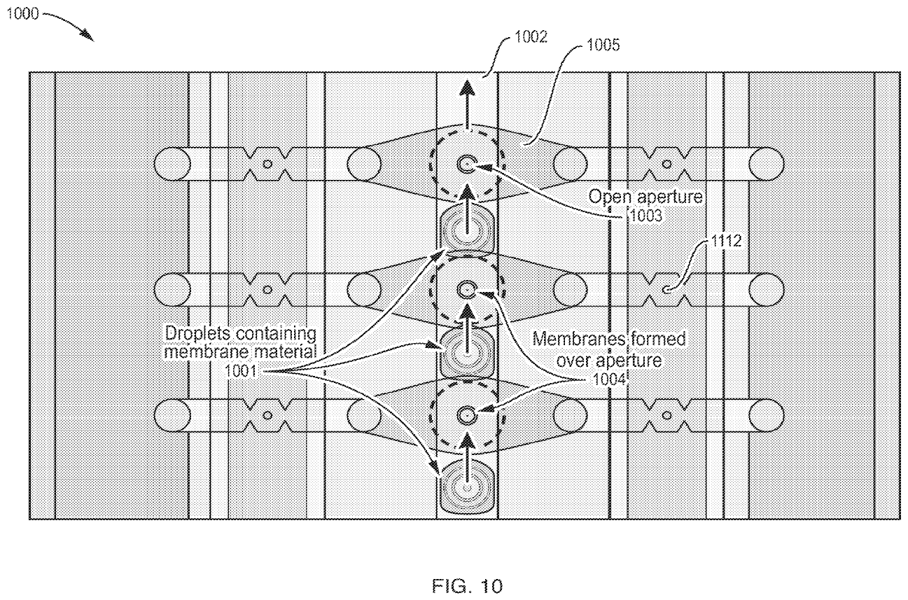

Another embodiment relates to a method to form membranes in a device as described in embodiments disclosed above. Organic and biological membranes are produced using a channel that flows the membrane material over the aperture supports in the shared channel, positioned above the recording chambers until a planar membrane is formed. These membrane forming materials such as lipids and polymers, are often dissolved in organic solvents. As shown in FIG. 10, described below, the solvent containing the material is flown over the apertures in the shared channel, in bulk form or as droplets suspended in a solution. A membrane is then formed at the interface of solvent and aperture. A control system may determine the flow of membrane materials during the membrane formation process, flowing the material through the shared channel as needed. Once membranes are formed, excess material may be flown out of the channels.

FIG. 10 is an image 1000 depicting formation of membrane via droplet flow in the shared channel 1002. Droplets of solvent or other fluids 1001 containing membrane material are flown through the shared channel 1002 over open apertures 1003. Membrane 1004 is then formed at the interface between the droplet and aperture 1003. This droplet flow can take place regardless of whether recording chambers 1005 are isolated.

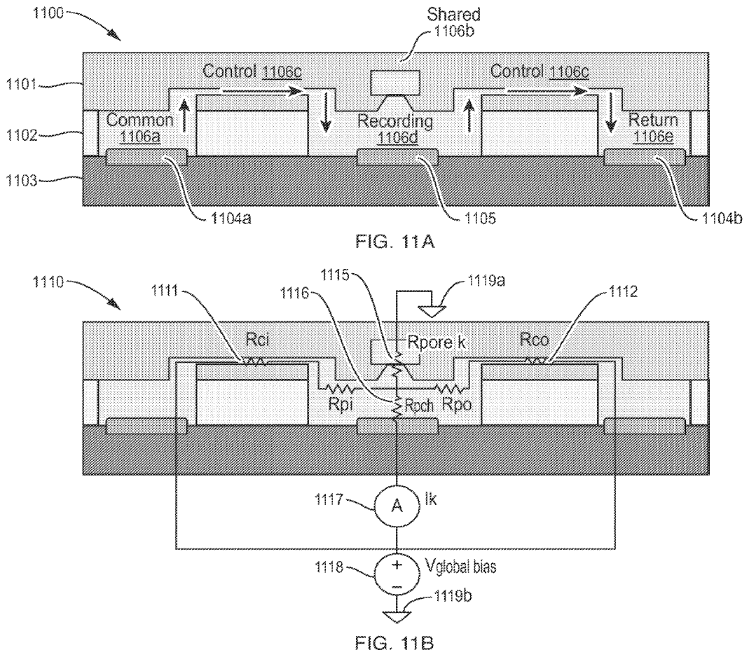

FIGS. 11A and 11B are lateral cross sections 1100 and 1110 respectively, of a resistively insulated device and the equivalent electrical circuit, according to an embodiment. The fluidic device includes dry film resist 1101, and the fluidic channels are contained within the channel walls 1102 which is positioned above the bottom substrate 1103. This embodiment of the fluidic device includes resistive insulation of recording chambers 1106d within an array from the common inflow 1106a and common return channels 1106e through the use of long and thin control channels 1106c, wherein the resistance of the control channels 1106c (R_{ci} 1111 and R_{co} 1112) is substantially larger than the resistance (R_{pch} 1116) between a pore and the recording electrode 1105 in the recording chamber 1106d, enforcing most of the current Iₖ 1117 through the pore to flow to the recording electrode 1105. For any particular design of the device, this fraction is a known ratio which may be corrected for in subsequent signal processing stages.

This embodiment maintains the physical fluid connection in the channels and avoids the use of control valves or sealing fluids. In this embodiment, the electrodes 1104a and 1104b within the common inflow 1106a and common return 1106e channels are held at a similar voltage to the recording electrode 1105 to produce no unwanted or corrupting current between them. Therefore, all recording electrodes 1105 of an array would be held at the same voltage (V_{global bias} 1118). The recorded current through the recording electrode 1105 would be slightly smaller than the total current through the pore having a resistance R_{poreK} 1115, where the difference is the current through the control channels. The fabrication of this embodiment relies on the aspect ratio (length/cross-sectional area) of the control channels 1106c, which determine the resistances (R_{ci} 1111, R_{co} 1112), to be substantially larger than the aspect ratio of the recording channel 1106d (which determines the recording channel 1106d resistance R_{pch} 1116).

In another embodiment, the resistive control channel 1106c may be fabricated to have varying cross-sectional profile, which may include a plurality of wide and narrow regions. The variations in cross-sectional profile may be implemented to achieve desired electrical resistance 1111 and 1112 and fluid flow resistance. In another embodiment, if recording chambers are designed for operation at different voltages, the resistance of control channels R_{ci} 1111 and R_{co} 1112 may be substantially larger than the resistance 1115 of a pore. This may be achieved through fabrication of very long and very thin control channels 1106c. In these embodiments, the electrical equivalency circuit described herein may also be connected to ground at 1119a and 1119b.

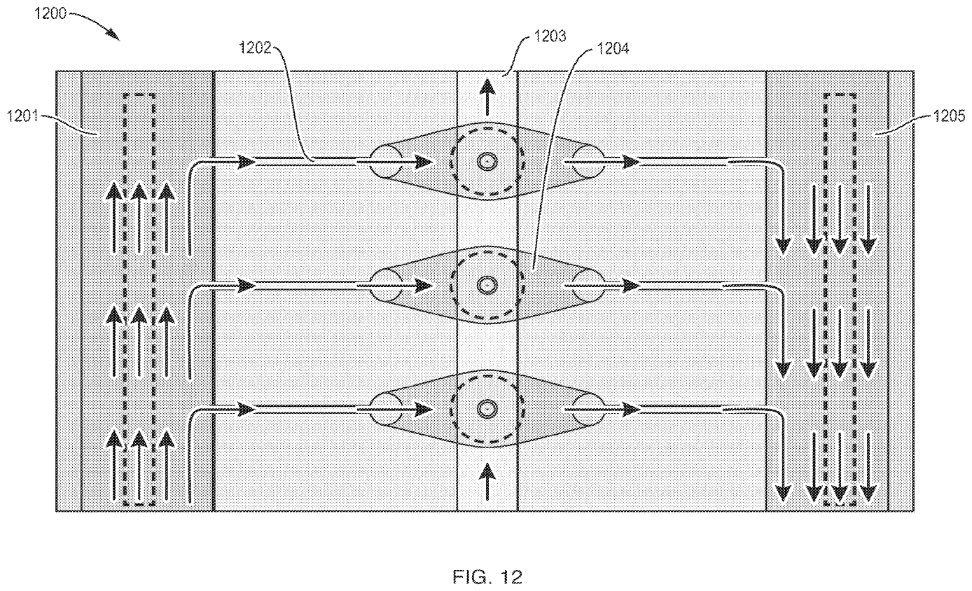

FIG. 12 is a top view 1200 of the fluidic device of the embodiments disclosed in reference to FIGS. 11A and 11B. Fluid flows from the common inflow channel 1201 through the thin control channels 1202 into the recording chamber 1204 and the shared channel 1203 and through to the common return channel 1205.

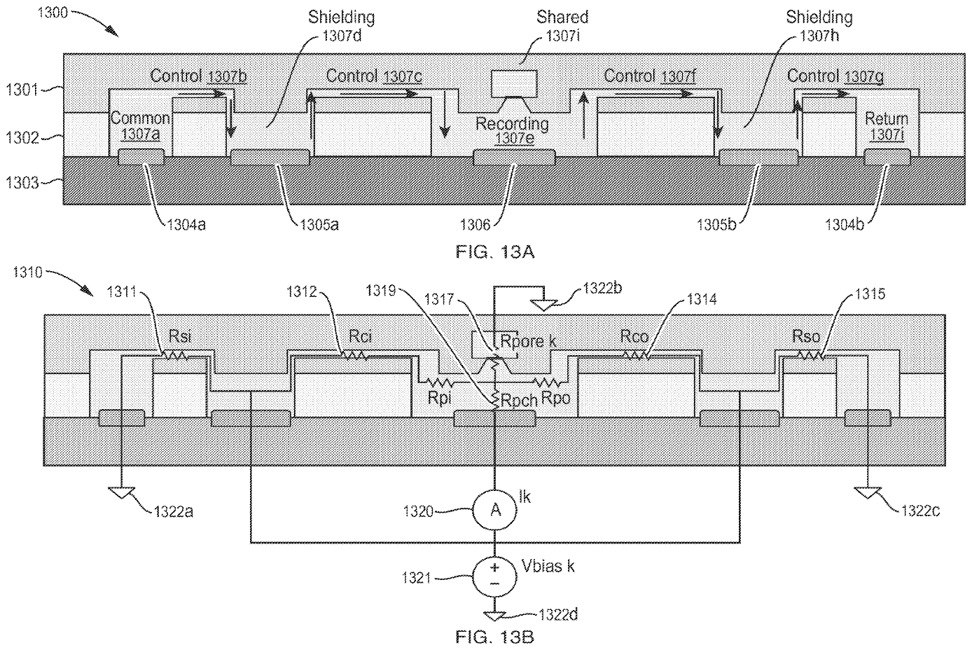

FIGS. 13A and 13B are lateral cross sections 1300 and 1310 respectively, of a resistively insulated device and the equivalent electrical circuit wherein the recording chambers may be at different voltages, according to an embodiment. The fluidic device includes dry film resist 1301, and the fluidic channels are contained within the channel walls 1302 which is positioned above the bottom substrate 1303. In this embodiment, to allow for operation of recording chambers 1307d, 1307e, 1307h, and 1307i at different voltages (V_{biask} 1321; wherein k is a chamber number within the array), a recording chamber resistance R_{pch} 1319, a guard buffer electrode 1305a, 1305b (also known as a shield electrode) may intersect the control channel 1307b, 1307c, 1307f, 1307g in a shielding chamber 1307d, 1307h. The guard buffer electrode relating to a particular chamber of the array is held at the same voltage 1321 (V_{biask}) as the recording electrode of that particular chamber, thereby electrically insulating different channels in the array from each other and from the common inflow 1307a and common return 1307i channels. The guard buffer electrodes 1305a and 1305b shall have low internal impedance to effectively shield the recording chambers 1307e from the common inflow 1307a and common return channels 1307i.

Similar to the embodiment disclosed in relation to FIG. 11A and FIG. 11B, the majority of the current Iₖ 1320 through the pore flows to the recording electrode 1306 and a small fraction of the current flows to the shield electrode 1305a and 1305b. The fraction of current flowing to the shield electrode 1305a and 1305b is dictated by the ratio of recording chamber 1307e resistance (between the pore and the recording electrode) R_{poreK} 1317 and the control channel resistance Rₛᵢ 1311, R_{ci} 1312, R_{co} 1314, and Rₛₒ 1315. The common inflow 1307a and common return 1307i channels connect through resistive channels (having resistance values of Rₛᵢ 1311 and Rₛₒ 1315, respectively) to the shield electrodes 1305a and 1305b. In some embodiments, the common inflow 1307a and common return 1307i channels may contain electrodes 1304a and 1304b set at any voltages. The current flow between the shield 1307d and 1307h and the common inflow 1307a and common return 1307i channels does not hinder the recording of nanopore current. In these embodiments, the electrical equivalency circuit described herein may also be connected to ground at 1322a-d. In all example embodiments described herein which use resistive control channels, the absence of mechanical insulation of channels through moving sealing membranes or fluids simplifies both fabrication and operation of the devices.

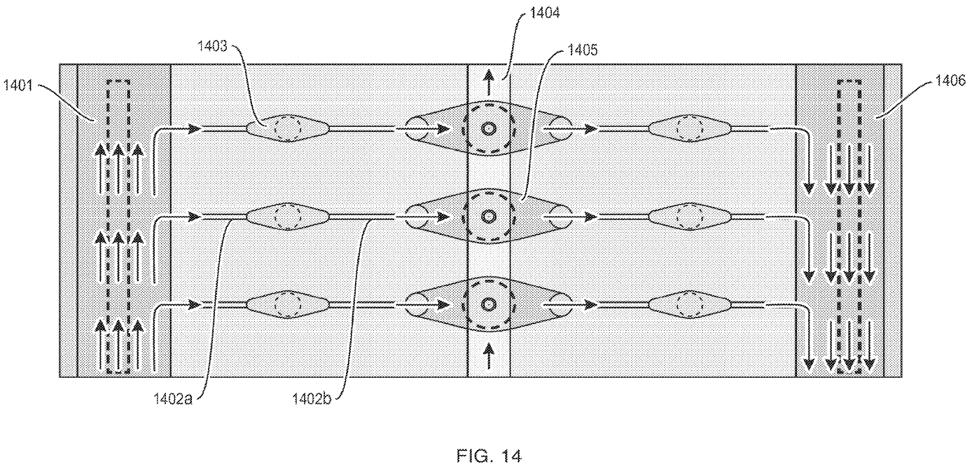

FIG. 14 is a top view 1400 of the fluidic device of the embodiments disclosed in reference to FIGS. 13A and 13B. Fluid flows from the common inflow channel 1401 through the thin control channel 1402a into the shielding chamber 1403, into the recording chamber 1405 and the shared channel 1404 and through to the common return channel 1406a.

While example embodiments have been particularly shown and described, it will be understood by those skilled in the art that various changes in form and details may be made therein without departing from the scope of the embodiments encompassed by the appended claims.

What is claimed is:

1. A pore sensing fluidic device, comprising:
a structure defining a common inflow channel, a shared channel with a plurality of apertures, a plurality of recording chambers, a common return channel, and a plurality of control channels arranged to support a fluid flow within the structure, the plurality of control channels connecting the common inflow channel to the shared channel and the plurality of recording chambers;
a sealer configured to isolate the plurality of recording chambers from the plurality of control channels;
a membrane formed and coupled to each of the plurality of apertures, and a pore formed in each membrane; and
a sensor, coupled to each of the plurality of apertures, configured to produce a representation of flow through each of the plurality of membrane pores.

2. The pore sensing fluidic device of claim 1, wherein the sealer includes the plurality of control channels configured to have an electrical resistance higher than an electrical resistance of each of the plurality of recording chambers.

3. The pore sensing fluidic device of claim 2, further comprising an electrode placed within each of the plurality of control channels, at least a subset of the electrodes configured to electrically insulate a recording chamber of the plurality of recording chambers.

4. The pore sensing fluidic device of claim 3, wherein the at least a subset of electrodes placed within each of the plurality of control channels is further configured to have a voltage equal to a voltage in the insulated recording chamber.

5. The pore sensing fluidic device of claim 1, wherein the sealer comprises a pressurized substance in combination with a sealing membrane, the pressurized substance forcing the sealing membrane to enter at least a subset of the plurality of control channels and isolate fluid flow from the shared channel and at least a subset of the plurality of recording chambers, the plurality of recording chambers to the common inflow channel, and the common return channel.

6. The pore sensing fluidic device of claim 1, wherein the sealer comprises a pressurized substance, the pressurized substance configured to enter at least a subset of the plurality of control channels and isolate fluid flow from the shared channel and at least a subset of the plurality of recording chambers to the common inflow channel and the common return channel.

7. The pore sensing fluidic device of claim 6, wherein the pressurized substance is non-soluble, non-conductive, and incapable of mixing with the fluid flow.

8. The pore sensing fluidic device of claim 1, wherein the representation of flow through the membrane pores is a representation of a rate of the flow, a representation of a measurement of a substance in the flow, or a combination thereof.

9. The pore sensing fluidic device of claim 8, wherein the measurement of the substance in the flow includes representations of ionic flow, representations of molecules in the flow, or a combination thereof.

10. The pore sensing fluidic device of claim 8, wherein the representation is processed by an external device configured to analyze the representation of a rate of flow, analyze a representation of the measurement of the substance in the flow, or a combination thereof.

11. The pore sensing fluidic device of claim 1, further comprising the plurality of membranes formed on and coupled to each of the plurality of apertures having (i) an electrical resistance being infinite before the membrane comprises a pore, or (ii) the electrical resistance being determined by an ability of ions to flow through the pore in the membrane of the plurality of membranes.

12. The pore sensing fluidic device of claim 1, wherein the pore is formed in the membrane by introducing a fluid solution containing a protein into: (i) the shared channel, or (ii) each recording chamber of the plurality of recording chambers.

13. The pore sensing fluidic device of claim 1, further comprising:
a shared channel input fluidic port configured to support fluid flow into the shared channel, and an output fluidic port configured to support fluid flow from the shared channel;
a common inflow channel input fluidic port configured to support fluid flow into the common inflow channel, and an output fluidic port configured to support fluid flow from the common inflow channel; and
a common return channel input fluidic port configured to support fluid flow into the plurality of recording chambers, and an output fluidic port configured to support fluid flow from the common return channel.

14. A method of constructing a pore sensing fluidic device, the method comprising:
defining, via a structure, a common inflow channel, a shared channel with a plurality of apertures, a plurality of recording chambers, a common return channel, and a plurality of control channels arranged to support a fluid flow within the structure, the plurality of control channels connecting the common inflow channel to the shared channel and the plurality of recording chambers;
associating a sealer with a plurality of control channels in an arrangement to enable the sealer to be operable to selectively isolate the plurality of recording chambers from the plurality of control channels;
forming and coupling a membrane to each of the plurality of apertures, and inserting a pore into each membrane; and
coupling a sensor to each of the plurality of apertures and configuring the sensor to produce a representation of flow through each of the plurality of membrane pores.

15. The method of constructing a pore sensing fluidic device of claim 14, wherein the plurality of control channels are configured to have an electrical resistance higher than an electrical resistance of each of the plurality of recording chambers.

16. The method of constructing a pore sensing fluidic device of claim 14, further comprising placing an electrode within each of the plurality of control channels, at least a subset of the electrodes configured to electrically insulate a recording chamber of the plurality of recording chambers from at least one other chamber.

17. The method of constructing a pore sensing fluidic device of claim 16, wherein the at least a subset of electrodes placed within each of the plurality of control channels is further configured to have a voltage being equal to a voltage in the insulated recording chamber.

18. The method of constructing a pore sensing fluidic device of claim 14, wherein the sealer comprises a pressurized substance in combination with a sealing membrane, the pressurized substance forcing the sealing membrane to enter at least a subset of the plurality of control channels and isolate fluid flow from the shared channel and at least a subset of the plurality of recording chambers, the plurality of recording chambers to the common inflow channel, and the common return channel.

19. The method of constructing a pore sensing fluidic device of claim 14, wherein the sealing and isolating includes a pressurized substance, the pressurized substance configured to enter at least a subset of the plurality of control channels and isolate fluid flow from the shared channel and at least a subset of the plurality of recording chambers to the common inflow channel and the common return channel.

20. The method of constructing a pore sensing fluidic device of claim 19, wherein the pressurized substance is non-soluble, non-conductive, and incapable of mixing with the fluid flow.

21. The method of constructing a pore sensing fluidic device of claim 14, wherein the representation of flow through the membrane pores is a representation of a rate of the flow, a representation of a measurement of a substance in the flow, or a combination thereof.

22. The method of constructing a pore sensing fluidic device of claim 21, wherein the measurement of the substance in the flow includes representations of ionic flow, representations of molecules in the flow, or a combination thereof.

23. The method of constructing a pore sensing fluidic device of claim 21, further comprising processing the representation by an external device configured to analyze the representation of a rate of flow, analyze a representation of the measurement of the substance in the flow, or a combination thereof.

24. The method of constructing a pore sensing fluidic device of claim 14, further comprising forming and coupling the plurality of membranes to each of the plurality of apertures, the membranes having (i) an electrical resistance being infinite before the membrane comprises a pore, or (ii) the electrical resistance being determined by an ability of ions to flow through the pore in the membrane of the plurality of membranes.

25. The method of constructing a pore sensing fluidic device of claim 14, further comprising forming the pore in the membrane by introducing a fluid solution containing a protein into: (i) the shared channel, or (ii) each recording chamber of the plurality of recording chambers.

26. The method of constructing a pore sensing fluidic device of claim 14, further comprising:
forming a shared channel input fluidic port to support fluid flow into the shared channel, via the shared channel input fluidic port, and forming a shared channel output fluidic port for supporting fluid flow from the shared channel via the shared channel output fluidic port;
forming a common channel input fluidic port to support fluid flow into the common channel via the common channel input fluidic port, and forming a common channel output fluidic port to support fluid flow from the common channel via the common channel output fluidic port; and
forming a recording chamber input fluidic port to support fluid flow into the recording chamber via a recording chamber input fluidic port and forming a recording chamber output fluidic port to support fluid flow from the recording chamber via the recording chamber output fluidic port.

27. A pore sensing fluidic device for ascertaining a molecular makeup of a substance, the fluidic device comprising:
means for isolating a portion of a fluid in a fluid flow, the portion being isolated in a chamber; and
means for producing a representation of ionic current of or molecules in the portion of the isolated fluid through a pore at the chamber.

28. A method of operating pore sensing fluidic device for ascertaining a molecular makeup of a substance, the method comprising:
isolating a portion of a fluid in a fluid flow, the portion being isolated in a chamber; and
producing a representation of ionic current of or molecules in the portion of the isolated fluid through a pore at the chamber.
